(19) United States
(12) Patent Application Publication
(10) Pub. No.: US 2026/0053238 A1
(43) Pub. Date: Feb. 26, 2026

(54) TECHNIQUES FOR FORMING GLASS DOMES FOR WEARABLE DEVICES

(71) Applicant: Oura Health Oy, Oulu (FI)

(72) Inventor: Jukka Tapani Mäkinen, Oulu (FI)

(21) Appl. No.: 19/307,942

(22) Filed: Aug. 22, 2025

Related U.S. Application Data
(60) Provisional application No. 63/745,420, filed on Jan. 15, 2025.
(60) Provisional application No. 63/687,213, filed on Aug. 26, 2024.

(51) Int. Cl. A44C 9/00; A61B 5/00; C03B 23/03

(52) U.S. Cl. CPC A44C 9/0053; A61B 5/0059; A61B 5/6826; C03B 23/0307; A61B 2560/0406

(57) ABSTRACT

Methods, systems, and devices for manufacturing a wearable device are described. Some techniques described herein may enable formation of dome-shaped protrusions for wearable devices from a glass material. For example, the glass may be formed into a dome shape and sealed to a housing of the wearable device to form a relatively more robust hollow dome-shaped protrusion that may result in relatively higher quality of measurements. In some examples, the dome-shaped protrusions may be formed from a sheet of glass that is curved into a molded shape or from an unmolded glass preform that is pressed into the molded shape. In some examples, the glass may be bonded with the housing of the wearable device during the molding process (e.g., with the housing forming a portion of the mold for the dome-shaped protrusion) or following the molding process.

### CROSS REFERENCE

The present Application for Patent claims priority to U.S. Provisional Patent Application No. 63/745,420 by Mäkinen et al., entitled “TECHNIQUES FOR FORMING GLASS DOMES FOR WEARABLE DEVICES,” filed January 15, 2025, and U.S. Provisional Patent Application No. 63/687,213 by Mäkinen et al., entitled “TECHNIQUES FOR FORMING GLASS DOMES FOR WEARABLE DEVICES,” filed August 26, 2024, assigned to the assignee hereof and expressly incorporated by reference herein.

### FIELD OF TECHNOLOGY

The following relates to wearable devices and data processing, including techniques for forming glass domes for wearable devices.

### BACKGROUND

Some wearable devices may be configured to collect physiological data from users via one or more light-emitting and light-receiving components.

### BRIEF DESCRIPTION OF THE DRAWINGS

FIG. 1 shows an example of a wearable device diagram that supports techniques for forming glass domes for wearable devices in accordance with aspects of the present disclosure.

FIG. 2 shows an example of a wearable device diagram that supports techniques for forming glass domes for wearable devices in accordance with aspects of the present disclosure.

FIG. 3 shows an example of a manufacturing diagram that supports techniques for forming glass domes for wearable devices in accordance with aspects of the present disclosure.

FIG. 4 shows an example of a manufacturing diagram that supports techniques for forming glass domes for wearable devices in accordance with aspects of the present disclosure.

FIGS. 5 and 6 illustrate examples of systems that support techniques for forming glass domes for wearable devices in accordance with aspects of the present disclosure.

FIG. 7 shows a flowchart illustrating methods that support techniques for forming glass domes for wearable devices in accordance with aspects of the present disclosure.

### DETAILED DESCRIPTION

Wearable devices can be configured to collect physiological data from users to provide users with more information regarding their overall health. The wearable devices may be configured to collect physiological data from the users via sensors (e.g., light-emitting diodes (LEDs), photodiodes) positioned within or beneath apertures of the wearable devices. In some examples, the apertures may be covered with a dome-shaped protrusion formed from an optically transparent material (e.g., epoxy, foil, an ultraviolet or heat curable material), which may enable relatively higher quality physiological measurements than if the dome was not present. In some examples, however, light may reflect from an interface between the skin and the optically clear material, which may result in a relatively lower quality of measurements due to a total internal reflection (TIR). To reduce the TIR, the dome-shaped protrusions may be hollow (e.g., air-filled). However, such hollow dome-shaped protrusions may be relatively less robust (e.g., less durable, more difficult to precisely manufacture) than solid (e.g., material-filled) dome-shaped protrusions, which may result in relatively more damage to the wearable device.

Accordingly, as described herein, dome-shaped protrusions for wearable devices may be formed from a glass material. For example, the glass may be formed into a dome shape and sealed to a housing of the wearable device to form a relatively more robust hollow dome-shaped protrusion that may result in relatively higher quality of measurements (e.g., as compared to some other optically transparent materials, such as epoxy). In some examples, the dome-shaped protrusions may be formed from a sheet of glass that is curved into a molded shape or from an unmolded glass preform that is pressed or vacuumed into the molded shape. In some examples, the glass may be bonded with the housing of the wearable device during the molding process (e.g., with the housing forming a portion of the mold for the dome-shaped protrusion) or following the molding process.

In some aspects, the dome-shaped protrusions may be formed from separate glass substrates (e.g., a separate sheet or preform for each dome-shaped protrusion). In such examples, each dome-shaped protrusion may be molded and sealed to the inner housing separately. Additionally, or alternatively, one or more dome-shaped protrusions may be formed from a same glass substrate (e.g., a single sheet or preform for one or more dome-shaped protrusions).

Aspects of the disclosure are illustrated by and described with reference to wearable device diagrams and manufacturing diagrams. Aspects of the disclosure are further described in the context of systems supporting physiological data collection from users via wearable devices and flowcharts that relate to techniques for forming glass domes for wearable devices.

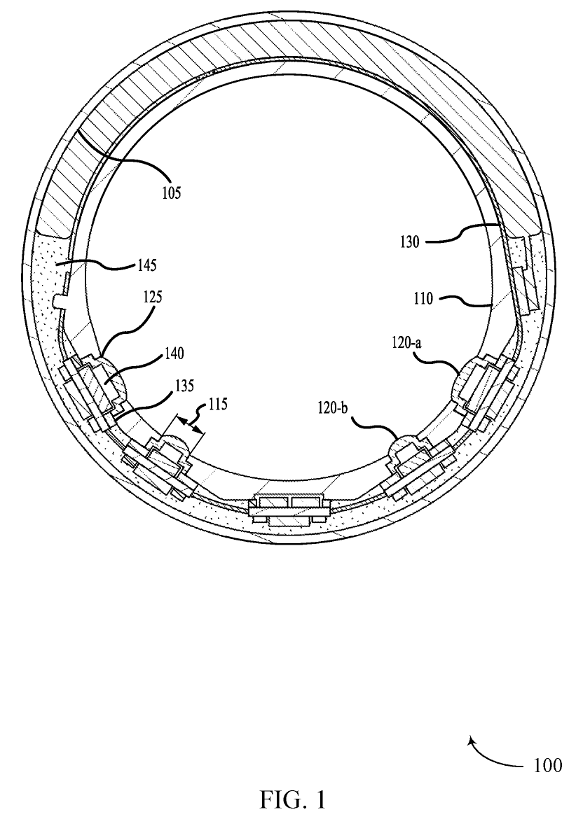

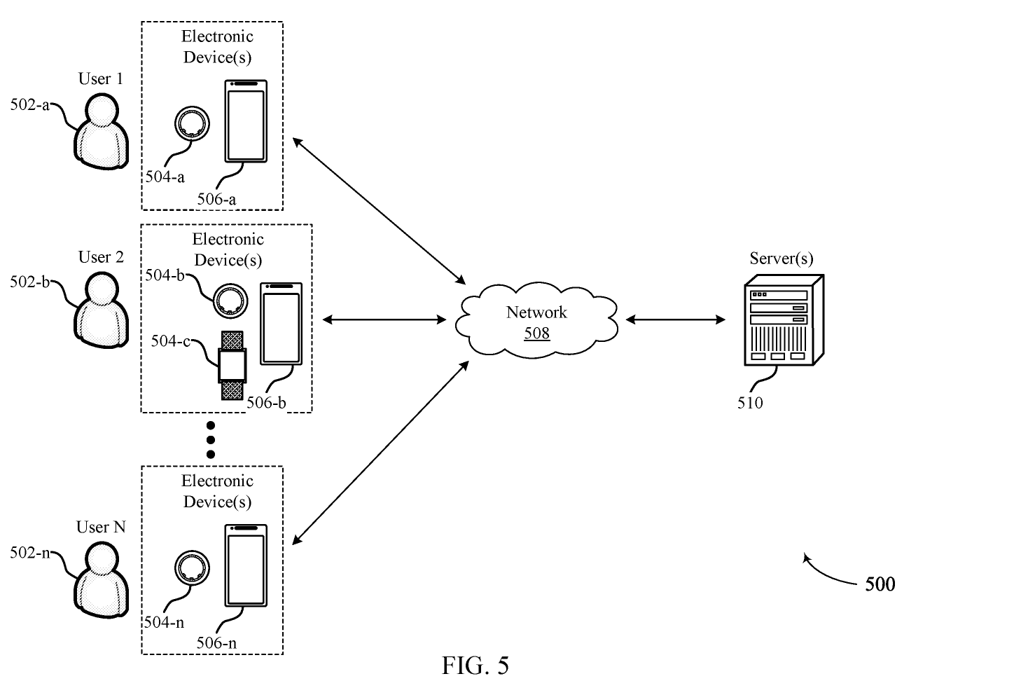

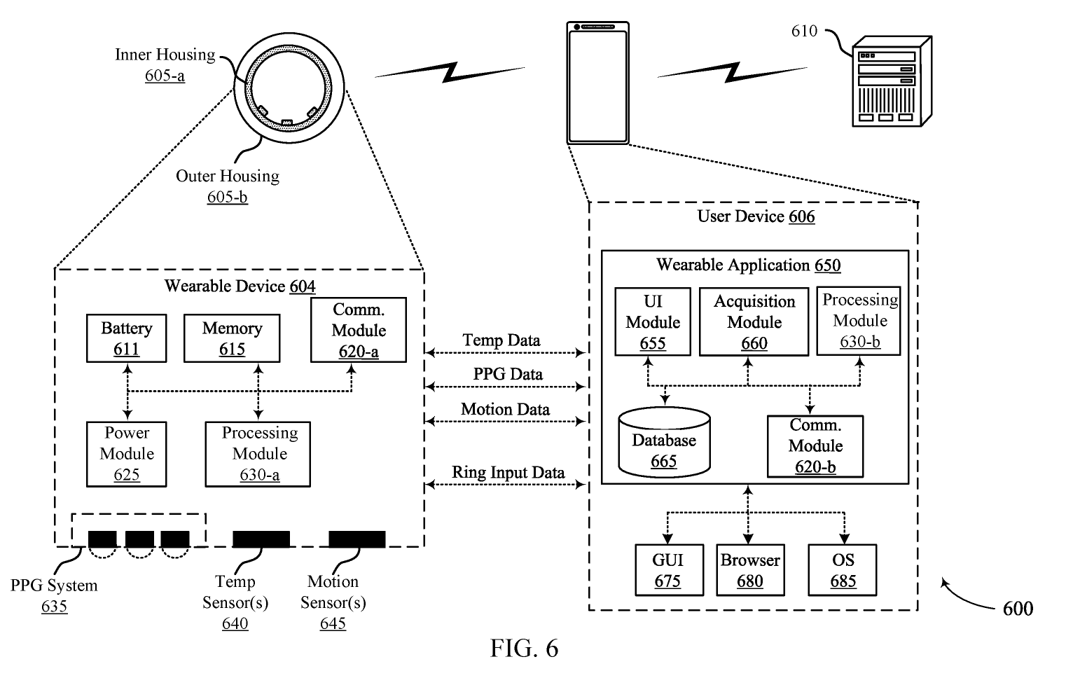

FIG. 1 shows an example of a wearable device diagram 100 that supports techniques for forming glass domes for wearable devices in accordance with aspects of the present disclosure. The wearable device diagram 100 may implement or may be implemented by aspects of the system 500 or the system 600. For example, the wearable device diagram 100 may illustrate a wearable device 604, which may be an example of the corresponding devices as described with reference to FIGS. 5 and 6

In some examples, as illustrated with reference to FIG. 6 , a wearable device (e.g., a wearable ring device 604, a wrist-worn wearable device 604) may include an outer housing 105 and an inner housing 110. The inner housing 110 and/or the outer housing 105 may include a metal material (e.g., stainless steel, titanium, or the like). The inner housing 110 and/or the outer housing 105 may house one or more electronic components 130 (e.g., a printed circuit board (PCB), a rechargeable battery, temperature sensors, photoplethysmogram (PPG) sensors such as one or more light-receiving components such as photodiodes (PDs) and one or more light-emitting components such as LEDs). The electronic components 130 may be attached to the inner housing 110 via attachment tape 135. In some examples, a space between the inner housing 110 and the outer housing 105 (e.g., a space that is not occupied by the electronic components 130) may be filled with a material (e.g., a filler epoxy 145).

In some examples, the wearable device may use the PPG sensors to collect physiological data associated with a user. For example, the PPG sensors may function by illuminating a tissue of a user (e.g., skin) via the light-emitting components and measuring an amount of light scattered back from pulsating blood vessels via the light-receiving components. In some examples, one or more dome-shaped protrusions 120 (e.g., dome-shaped protrusions 120-a over the light-receiving components and dome-shaped protrusions 120-b over the light-emitting components) may be designed to be formed on top of the light-emitting components and the light-receiving components to increase a signal quality by changing an incidence angle of light from the light-emitting components that enters into the tissue of the user to reduce a TIR associated with the light, which may reduce an amount of light that is input into the tissue of the user. For example, when light propagating inside optically clear material hits an interface between the material and another optical material with lower refractive index, the light can be reflected back into the lightguide if the angle of incidence is large enough.

In some examples, however, the light may reflect back from an interface between the dome-shaped protrusions 120 and the tissue of the user (e.g., or air between the dome-shaped protrusions and the tissue of the user), which may reduce optical efficiency and signal quality. Such lower PPG sensor optical efficiency may increase power consumption. Additionally, back-reflected light from the light-emitting components may increase an amount of internal stray light in the wearable device.

For example, skin may be relatively less smooth (e.g., with ridges and valleys) as compared to a material forming the dome-shaped protrusions 120. Accordingly, the ridges and valleys may make uneven contact to the surface of the dome-shaped protrusions 120. That is, there may be one or more points of contact between the dome-shaped protrusions 120, the ridges of the tissue, and the air filling the valleys between the ridges of the tissue. Additionally, if the tissue is pressed harder against an interface between the tissue and the dome-shaped protrusions 120, a surface area may increase as the ridges and valleys flatten out. Accordingly, a level of skin contact to the dome-shaped protrusions 120 and therefore an amount of TIR may depend on a fit of the wearable device. In some examples, relatively higher contact pressure may push blood out from vascular layers of the skin, which may result in a relatively weaker or stronger PPG signal, which may be different from a PPG signal associated with a relatively higher quality of measurements.

In some examples, an amount of light outcoupling from the light-emitting components may depend on a shape of the dome-shaped protrusions 120. For example, the dome-shaped protrusions 120 may have a height that reduces (e.g., minimizes) TIR at an interface between the skin and the dome-shaped protrusions 120. In some examples, the amount of light outcoupling may change relatively significantly with respect to dome shape. Accordingly, variation in shape of the dome-shaped protrusions 120 may result in signal level variation in PPG measurements.

In some examples, to reduce an amount of TIR, the dome-shaped protrusions 120 may be hollow (e.g., with an air cavity 140). For example, evenly thick optical layers (e.g., that are not in direct contact with the light-emitting components) may reduce an amount of light trapped inside the material forming the dome-shaped protrusions as a result of TIR, which may increase measurement efficiency, measurement quality, and battery life. In some examples, the dome-shaped protrusions 120 may be formed from a plastic or foil material, which may be manufactured by vacuum molding. Additionally, dome-shaped protrusions 120 that have a relatively more even thickness may reduce an amount of signal distortion related to deformations in manufacturing. That is, a tolerance associated with shaping the dome-shaped protrusions 120 may be relatively higher for hollow dome-shaped protrusions 120 as compared to non-hollow dome-shaped protrusions 120. Accordingly, a cost of manufacturing hollow dome-shaped protrusions 120 may be relatively reduced as compared to non-hollow dome-shaped protrusions 120.

However, such plastic or foil dome-shaped protrusions 120 may be relatively less robust than solid domes or dome-shaped protrusions formed from some other materials. For example, a plastic material may not block moisture from entering the wearable device, which may damage the electronic components 130. Additionally, some polymer materials may scratch relatively easier than some other materials, and in some examples, may soften in relatively lower temperatures than some other materials.

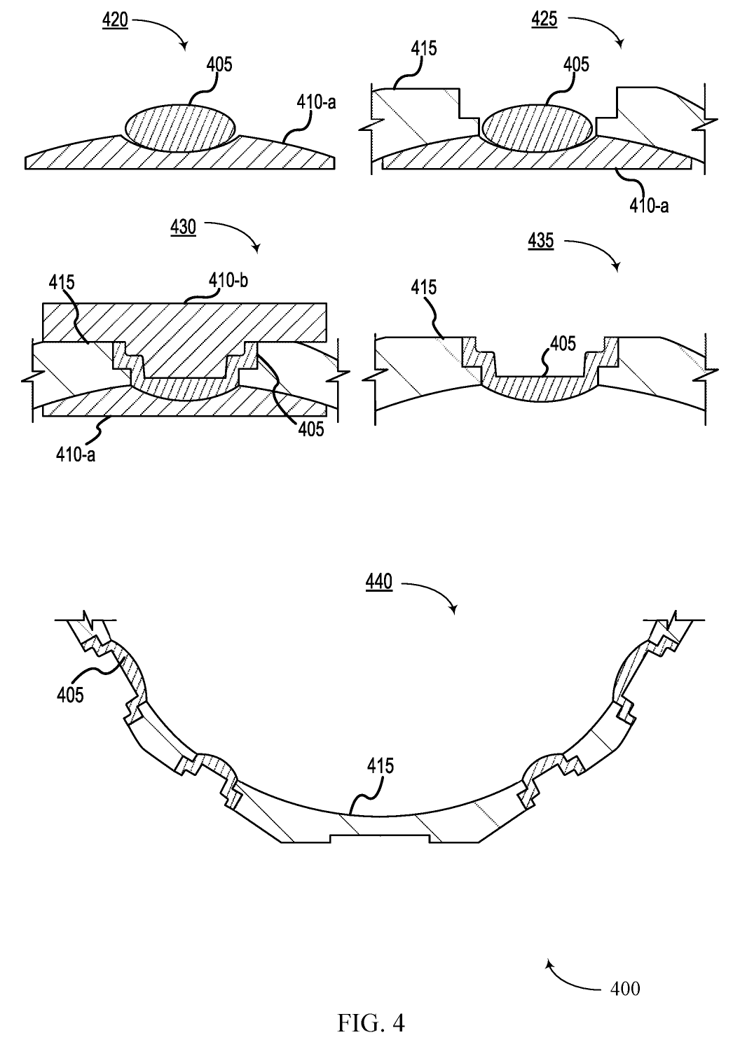

Accordingly, techniques described herein may enable a wearable device (e.g., wearable ring device) to be manufactured with dome-shaped protrusions 120 that are molded from a glass material (e.g., a glass material that may be optically similar to a plastic material of the dome-shaped protrusions 120). The glass material may be relatively more robust than some plastic materials (e.g., against moisture, scratching, and heat). Accordingly, the wearable device may include hollow glass dome-shaped protrusions 120 that may increase a light efficiency of the PPG sensors and accordingly increase a battery life and signal quality of the wearable device. The glass material may be attached and sealed to the inner housing 110 by directly molding or gluing them into one or more apertures 115 of the wearable ring device, as described herein with reference to FIGS. 4 and 5

In some examples, the glass dome-shaped protrusions 120 may be molded using thin sheet shaping. The glass dome-shaped protrusions 120 may be sealed to a metal material of the aperture 115 (e.g., a metallic inlet of the inner housing 110) using a glass-to-metal sealing procedure (e.g., by using an adhesive material such as glue or by hermetically sealing the glass dome-shaped protrusions 120 to the metal material). In some examples, the glass dome-shaped protrusions 120 may be coated in metal and welded to the inner housing 110.

In some examples, to seal the glass dome-shaped protrusions 120 onto the metal of the inner housing 110 (e.g., to form a hermetic sealing or glue seal 125), a chemical bond, compression bond, or both may be formed. In some examples, such a glass-metal seal may be formed using mechanical bonding (e.g., by a nature of roughened surfaces) where a surface roughness may result in a frictional force that may be overcome to separate the sealed portion. The glass material of the glass dome-shaped protrusions 120 may penetrate surfaces, pores, or cavities of the metal material, which may provide further surface area for interlocking the structures of the glass dome-shaped protrusions 120 and the metal inner housing 110. Additionally, or alternatively, the glass-metal seal may be formed via chemical bonding, which may refer to atomic or molecular bonds between the glass material and the metal material. For example, the metal material may be pre-oxidized to produce a bond between the glass material and the metal material.

In some examples, a seal (e.g., a glass-metal seal) may be matched or unmatched based on thermal expansion. For a matched seal, a coefficient of thermal expansion (CTE) of the glass material and a CTE of the metal material may be relatively similar. In such examples, the sealing may be a result of chemical bonding at the glass-metal interface. Such matched seals may be formed from metals, ceramics, or glasses. Matched seals may be categorized according to an expansion coefficient of the glass (e.g., and an expansion of the metal). For example, some glasses (e.g., boro-silicates) may be hard glasses and some glasses (e.g., soda glasses) may be soft glasses.

For an unmatched seal, the CTE of the metal material may be different from the CTE of the glass material. Unmatched seals may be categorized as ductile and compression seals. Ductile seals may be used to join glass to copper, stainless steel, or Kovar (e.g., due to a high CTE). The metal material may be thinned to comply with the glass material and accordingly to form a seal. Compression seals may be formed by a mechanical bond (e.g., by establishing a compressive hoop force) from the metal material onto the glass material. For example, the metal material may shrink around the sealing glass during production, which may result in a compressive force establishing the seal. Such compressive seals may depend on a compressive strength of the glass material (e.g., which may be relatively higher than a tensile strength of the glass material).

In some examples, the glass material and the metal material may be selected based on achieving a particular CTE for a given fabrication route and anticipated temperature range. For example, boro-silicate or lead-silicate may be used on copper alloys and stainless steels to form the seal. In some examples, the inner housing 110 may be a titanium material that may have a similar CTE as the glass component. The glass material may be applied as frits, paste, or a solid preform. The glass material may be heated (e.g., using electrical, RF, or flame heating) to soften the glass to the correct viscosity for joining with the metal material. In some examples, the glass material may be placed under pressure (e.g., to provide intimate contact with the metal material). In some examples, a protective or forming gas atmosphere may be used to promote wetting and adhesion between the glass material and the metal material, with an annealing stage included during cooling to inhibit development of residual stress.

In some examples, to produce a glass-metal chemical bond, a metal piece (e.g., the metal material) may be heated in an oxidizing atmosphere (e.g., to evaporate gas-producing species and remove hydrocarbons). The metal piece may be heated in a wet hydrogen atmosphere (e.g., to eliminate an oxide layer formed on the surface), which may produce a pure metal surface which may be oxidized. The metal material may undergo a controlled oxidation in air atmosphere to produce an adherent oxide for the glass material to dissolve and to which the glass material may bond. In some examples, the atmosphere, temperature, and time for oxidation may be controlled to result in a desired oxide layer thickness, uniformity, and oxide species to which the glass material may bond reliably. For example, a stainless steel metal material may form a chemical bond with the glass material via an intermediate layer of chromium-oxide and iron-oxide.

In some examples, the glass dome-shaped protrusions 120 may be on both of the inner housing 110 and the outer housing 105 of the wearable device. For example, the glass dome-shaped protrusions 120 on the outer housing 105 of the wearable ring device may be decorative (e.g., as jewels) or may serve as a window (e.g., for an indicator light or decorative lights on the wearable ring device, a solar cell configured to charge the rechargeable battery of the wearable ring device, ambient light sensors, gesture sensors). In some examples, the glass dome-shaped protrusions 120 may have a higher-quality feel than plastic dome-shaped protrusions 120, which may increase a value of the wearable device.

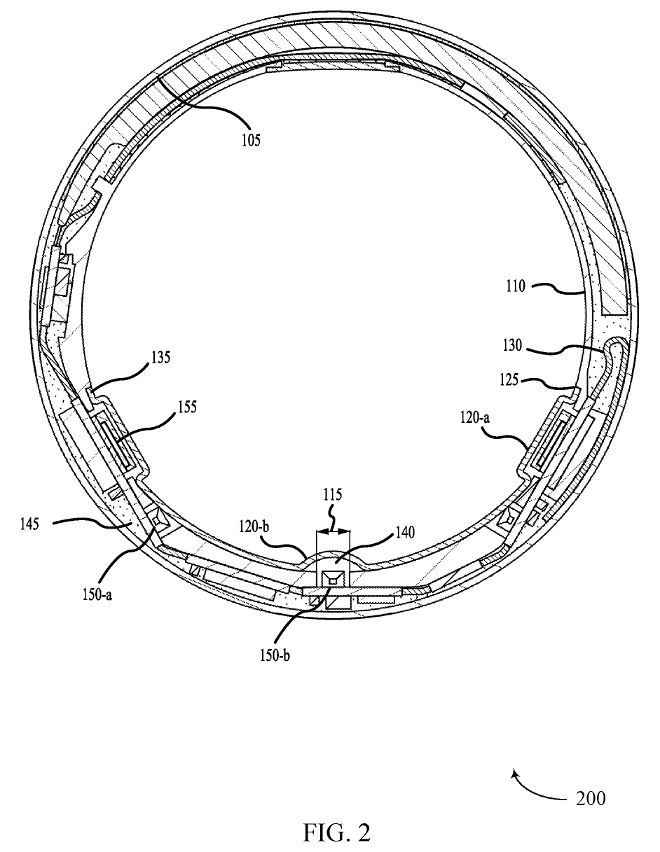

FIG. 2 shows an example of a wearable device diagram 200 that supports techniques for forming glass domes for wearable devices in accordance with aspects of the present disclosure. The wearable device diagram 200 may implement or may be implemented by aspects of the wearable device diagram 100, the system 500, or the system 600. For example, the wearable device diagram 100 may illustrate a wearable device 604, which may be an example of the corresponding devices as described with reference to FIGs. 5 and 6.

In some examples, as illustrated with reference to FIG. 6, a wearable device (e.g., a wearable ring device 604, a wrist-worn wearable device 604) may include an outer housing 105 and an inner housing 110. The inner housing 110 and/or the outer housing 105 may be a metal material (e.g., steel, titanium, or the like). The inner housing 110 and/or the outer housing 105 may house one or more electronic components 130 (e.g., a PCB, a rechargeable battery, temperature sensors, PPG sensors such as one or more light-receiving components 155 such as PDs and one or more light-emitting components 150 such as LEDs). The electronic components 130 may be attached to the inner housing 110 via attachment tape 135. In some examples, a space between the inner housing 110 and the outer housing 105 (e.g., a space that is not occupied by the electronic components 130) may be filled with a material (e.g., a filler epoxy 145).

In some examples, the wearable device may use the PPG sensors to collect physiological data associated with a user, as described with reference to FIGS. 1 and 6 In some examples, one or more dome-shaped protrusions 120 (e.g., dome-shaped protrusions 120-a over the light-receiving components 155 and dome-shaped protrusions 120-b over the light-emitting components 150) may be over the light-emitting components 150 and the light-receiving components 155 to increase a signal quality as described herein.

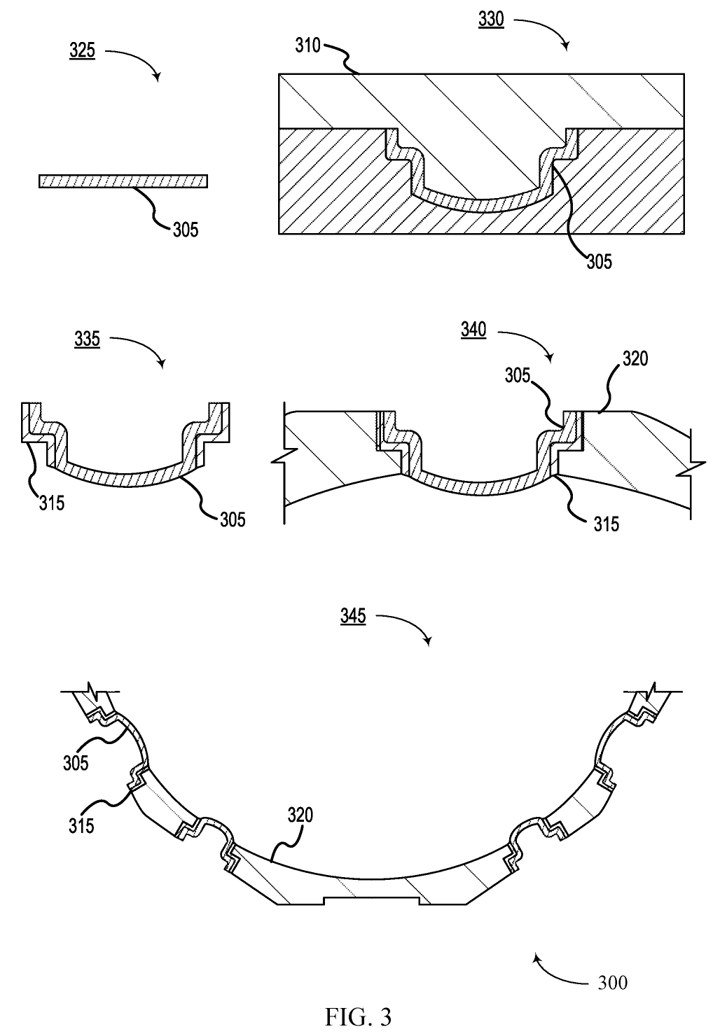

For example, as described with reference to FIG. 1 the dome-shaped protrusions 120 may be molded from a glass material (e.g., a glass material that may be optically similar to a plastic material of the dome-shaped protrusions 120). The glass material may be relatively more robust than some plastic materials (e.g., against moisture, scratching, and heat). Accordingly, the wearable device may include hollow glass dome-shaped protrusions 120 that may increase a light efficiency of the PPG sensors and accordingly increase a battery life and signal quality of the wearable device. The glass material may be attached and sealed to the inner housing 110 by directly molding or gluing them into one or more apertures 115 of the wearable ring device, as described herein with reference to FIG. 1, FIGS. 3 and 4. The dome-shaped protrusions 120 may be formed from a glass sheet or a glass preform, as described herein with reference to FIGS. 1,3 and 4

In some examples, as illustrated with reference to FIG. 1, each dome-shaped protrusion 120 may be formed from a separate glass substrate (e.g., sheet or preform). Additionally, or alternatively, as illustrated with reference to FIG. 2, one or more dome-shaped protrusions 120 may be formed from a same glass substrate. For example, a single glass substrate may be molded as described with reference to FIGS. 3 and or 4 to form dome-shaped protrusions that may cover multiple sensors. In such examples, the glass substrate may partially or fully cover one or more metal portions of the inner housing 110 (e.g., portions of the inner housing 110 forming the apertures 115). In some aspects, the glass substrate may form a portion of the inner housing 110.

In some aspects, one or more light-emitting components 150 may be located in one or more different locations along an inner circumference of the wearable device. For example, one or more light-emitting components 150-a configured to emit light of a first wavelength (e.g., green LEDs which may use relatively shorter optical paths) may be located relatively closer to one or more light-receiving components 155 as compared to one or more light-emitting components 150-b configured to emit light of a second wavelength (e.g., red or infrared LEDs which may use relatively longer optical paths).

In some examples, each light-emitting component 150-a may be located on a same printed circuit board (PCB) or printed wiring board (PWB) as a respective nearby light-receiving component 155 (e.g., the inner housing 110 results in light blocking between the light-emitting component 150-a and the light-receiving component 155). In some examples, a portion of the glass substrate covering the one or more light-emitting components 150-a may be relatively flatter (e.g., in line with a circle forming the inner circumference of the wearable device, such as a portion not formed into a dome-shaped protrusion 120) as compared to a portion of the glass substrate covering the one or more light-emitting components 150-b (e.g., a portion formed into a dome-shaped protrusion 120-b).

As an illustrative example, the wearable device may include multiple (e.g., two) light-emitting components 150-a and a single light-emitting component 150-a (e.g., as illustrated with reference to FIG. 2). Additionally, or alternatively, the wearable device may include multiple (e.g., two) light-emitting components 150-a and multiple (e.g., two) light-emitting components 150-a (e.g., each housed within a separate dome-shaped protrusion 120), which may result in a relatively higher signal quality as compared to a single light-emitting component 150-a. In some examples, the wearable device may include multiple light-receiving components 155 configured to measure light via an optical channel between the light-receiving component 155 and each light-emitting component 150.

Additionally, or alternatively, one or more cavities of the wearable device may house multiple light-emitting components 150. For example, a cavity housing the light-emitting component 150-a may house an additional light-emitting component 150 configured to emit light of a third wavelength (e.g., yellow LEDs) and a cavity housing the light-emitting component 150-b may house an additional light-emitting component 150-b. Additionally, or alternatively, the wearable device may include one or more additional cavities housing one or more additional light-emitting components 150 configured to emit light of a fourth wavelength (e.g., vertical-cavity surface-emitting lasers (VCSELs) configured to emit infrared light). In such examples, each cavity may include reflector optics configured to separate optical channels associated with each light-emitting component 150. Accordingly, the wearable device may include relatively more light-emitting components 150 (e.g., and multiple light-emitting components 150 in a same space, such as within a same cavity or on a same PWB of the wearable device). Such techniques may enable the wearable device to control light emission patterns, improve accuracy of measurements, and/or enable the wearable device to perform additional measurements (e.g., spectral measurements such as hydration, hemoglobin levels, lipids, ethanol, glucose, and the like).

In some aspects, multiple light-emitting components 150 in various locations across the inner circumference of the wearable device may form multiple optical channels between the light-emitting components 150 and the light-receiving components 155. For example, a multiple-LED approach (e.g., with three or more light-emitting components 150) may enable each light-receiving component 155 to measure multiple optical channels between the respective light-receiving component 155 and each light-emitting component 150. The wearable device may accordingly select an optical channel via which to measure the light (e.g., from multiple optical channels of varying length), which may enable the wearable device to select an optical channel with relatively higher quality of measurements. Accordingly, separate light-emitting components 150 that are varying distances from each light-receiving component 155 may result in a relatively higher measurement quality as compared to wearable devices with multiple light-emitting component chips in a same package (e.g., housed within a same dome-shaped protrusion). Additionally, such separate light-emitting components 150 may be relatively smaller than multiple light-emitting component chips in a same package, which may enable relatively more flexible placement of the light-emitting components.

In some examples, each light-receiving component 155 may be a chip-level photodiode. Such chip-level photodiodes may include one or more relatively more light-sensitive areas, which may enable relatively more measurement collection as a result of receiving more signal. In some examples, each light-emitting component 150 may be a chip-level LED. Such chip-level LEDs may enable the wearable device to control light emission patterns, which may enable collection of relatively more physiological measurements.

In some aspects, the wearable device may be relatively thinner than some other wearable devices (e.g., wearable devices with non-hollow dome-shaped protrusions 120). For example, the electronic components 130 may be relatively closer to an inner surface of the wearable device (e.g., such that the light-emitting components 150 and/or the light-receiving components 155 are located within the dome-shaped protrusions above the inner housing 110), which may increase user comfort associated with wearing the wearable device and a cost of manufacturing the wearable device.

In some examples, the glass dome-shaped protrusions 120 may be on both of the inner housing 110 and the outer housing 105 of the wearable device. For example, the glass dome-shaped protrusions 120 on the outer housing 105 of the wearable ring device may be decorative (e.g., as jewels) or may serve as a window (e.g., for an indicator light or decorative lights on the wearable ring device, a solar cell configured to charge the rechargeable battery of the wearable ring device, ambient light sensors, gesture sensors). In some examples, the glass dome-shaped protrusions 120 may have a higher-quality feel than plastic dome-shaped protrusions 120, which may increase a value of the wearable device.

FIG. 3 shows an example of a manufacturing diagram 300 that supports techniques for forming glass domes for wearable devices in accordance with aspects of the present disclosure. The manufacturing diagram 300 may implement or may be implemented by aspects of the wearable device diagram 100, the wearable device diagram 200, the system 500, or the system 600. For example, the manufacturing diagram 300 may illustrate a method for manufacturing a wearable device 604, which may be an example of the corresponding devices as described with reference to FIGs. 5 and 6.

The manufacturing diagram 300 may illustrate a method of manufacturing glass dome-shaped protrusions of a wearable device, as described with reference to FIGS. 1 and 2. The manufacturing diagram 300 may illustrate an example in which one or more glass dome-shaped protrusions are formed separately from the metal inner housing of the wearable device and later attached or sealed to metal inlet apertures of the metal inner housing.

In some examples, at 325, a glass sheet 305 (e.g., a thin sheet of glass) may be cut to a size that may fit into a mold 310 to mold the glass sheet 305 into one or more dome shapes (e.g., a shape of the dome-shaped protrusions). For example, the mold may form the glass into a shape of a single dome-shaped protrusion (e.g., as illustrated with reference to FIG. 1) or into a shape of a portion of an inner housing of the wearable device including multiple dome-shaped protrusions (e.g., as illustrated with reference to FIG. 2). At 330, the glass sheet 305 may be placed into the mold 310. In some examples, a gas surrounding the glass sheet 305 may be adjusted for control of chemical reactions. The glass sheet 305 may be heated (e.g., electronically, via IR lamps, via a flame) such that it may be molded by external forces. In some examples, a vacuum may pull the glass into a form of the mold 310 (e.g., via vacuum-assisted slumping, by evacuating gas in a chamber of the mold 310). Additionally, or alternatively, the glass sheet 305 may be formed to the mold 310 using pressure.

The glass sheet 305 may be stable (e.g., may remain in the shape of the mold 310) when the glass sheet 305 is cooled down. In some examples, the glass sheet 305 may be cooled with nitrogen gas (e.g., N₂). In some examples, vacuum-assisted slumping may achieve relatively high forming rates with small surfaces for relatively thin materials (e.g., the glass sheet 305). Additionally, vacuum deep drawing may achieve a relatively small bending radius and relatively high aspect ratio more accurately than some other glass molding techniques.

At 335, the glass sheet may be removed from the mold 310 and an adhesive material 315 (e.g., glue) may be applied to the glass sheet. At 340, the glass sheet may be coupled to an inner housing 320 of a wearable device (e.g., via curing the glue material). Additionally, or alternatively, the glass sheet 305 may be coupled to the inner housing via taping, overmolding, or glass metal coating and welding. Additionally, or alternatively, a hermetic seal may be formed by creating controlled oxide layers on the metal material, which may enable proper conditions for the glass material to dissolve and form a chemical bond with the metal material.

At 345, the molding, adhering, and coupling process may be repeated one or more times to form multiple dome-shaped protrusions of the wearable device from glass sheets 305 that are sealed to the inner housing 320 via the adhesive material 315 (e.g., as illustrated with reference to FIG. 1). Additionally, or alternatively, the molding process described herein may form multiple dome-shaped protrusions from a single sheet of glass, and a single molding, adhering, and coupling process may form the multiple dome-shaped protrusions of the wearable device (e.g., as illustrated with reference to FIG. 2). In some examples, the glass sheet 305 may be coated in a metallic coating, which may increase a signal quality associated with light measured via light-emitting components and light-receiving components housed in the dome-shaped protrusions. A full wearable device may be assembled by coupling one or more electronic components to the inner housing 320, coupling an outer housing to the inner housing 320, and filling any remaining internal space of the wearable device with an epoxy filler.

FIG. 4 shows an example of a manufacturing diagram 400 that supports techniques for forming glass domes for wearable devices in accordance with aspects of the present disclosure. The manufacturing diagram 400 may implement or may be implemented by aspects of the wearable device diagram 100, the wearable device diagram 200, the manufacturing diagram 300, the system 500, or the system 600. For example, the manufacturing diagram 400 may illustrate a method for manufacturing a wearable device 604, which may be an example of the corresponding devices as described with reference to FIGs. 5 and 6.

The manufacturing diagram 400 may illustrate a method of manufacturing glass dome-shaped protrusions of a wearable device, as described with reference to FIGS. 1 and 2. The manufacturing diagram 400 may illustrate an example in which a glass dome-shaped protrusion is formed and sealed to the metal inner housing of the wearable device in a single process.

In some examples, at 420, a glass preform 405 may be placed into a bottom portion 410-a of a mold 310 that is shaped to mold the glass preform 405 into one or more dome shapes (e.g., a shape of the dome-shaped protrusions). For example, the mold may form the glass into a shape of a single dome-shaped protrusion (e.g., as illustrated with reference to FIG. 1) or into a shape of a portion of an inner housing of the wearable device including multiple dome-shaped protrusions (e.g., as illustrated with reference to FIG. 2). At 425, an inner housing 415 of a wearable device may be placed to form side walls of the mold (e.g., around the glass preform 405). At 430, a top portion 410-b of the mold may be placed above the glass preform 405. In some examples, a gas surrounding the glass preform 405 may be adjusted for control of chemical reactions. The glass preform 405 may be heated (e.g., electronically, via IR lamps, via a flame) such that it may be molded by external forces. In some examples, the metal material of the inner housing 415 may be heated prior to forming, which may enable compression sealing with the glass preform 405 (e.g., depending on the metal of the inner housing 415).

In some examples, a vacuum may pull the glass into a form of the mold 410 (e.g., via vacuum-assisted slumping by evacuating gas in a chamber of the mold 410). Additionally, or alternatively, the glass preform 405 may be formed to the mold using pressure. In some examples, the inner housing 415 may include mechanical features (e.g., mechanical locking features) that may be filled with glass during the pressure forming. Such techniques may result in a relatively stronger holding pressure due to interlocking structures forming a compressive force. Additionally, or alternatively, the glass preform 405 may be coupled to the inner housing 415 via taping, overmolding, or glass metal coating and welding. Additionally, or alternatively, a hermetic seal may be formed by creating controlled oxide layers on the metal material (e.g., by controlling a gas forming the atmosphere in the mold), which may enable proper conditions for the glass material to dissolve and form a chemical bond with the metal material.

The glass preform 405 may be stable (e.g., may remain in the shape of the mold) when the glass preform 405 is cooled down. In some examples, the glass preform 405 may be cooled with nitrogen gas (e.g., N₂). At 435, the glass preform 405 and the inner housing 415 may be removed from the mold. At 440, the molding and coupling process may be repeated one or more times to form multiple dome-shaped protrusions of the wearable device from glass preforms 405 that are sealed to the inner housing 415 via chemical or compressive sealing (e.g., as illustrated with reference to FIG. 1). Additionally, or alternatively, the molding process described herein may form multiple dome-shaped protrusions from a single glass preform 405, and a single molding, adhering, and coupling process may form the multiple dome-shaped protrusions of the wearable device (e.g., as illustrated with reference to FIG. 2). In some examples, the glass preform 405 may be coated in a metallic coating, which may increase a signal quality associated with light measured via light-emitting components and light-receiving components housed in the dome-shaped protrusions. A full wearable device may be assembled by coupling one or more electronic components to the inner housing 415, coupling an outer housing to the inner housing 415, and filling any remaining internal space of the wearable device with an epoxy filler.

FIG. 5 illustrates an example of a system 500 that supports techniques for forming glass domes for wearable devices in accordance with aspects of the present disclosure. The system 500 includes a plurality of electronic devices (e.g., wearable devices 504, user devices 506) that may be worn and/or operated by one or more users 502. The system 500 further includes a network 508 and one or more servers 510.

The electronic devices may include any electronic devices known in the art, including wearable devices 504 (e.g., ring wearable devices, watch wearable devices, etc.), user devices 506 (e.g., smartphones, laptops, tablets). The electronic devices associated with the respective users 502 may include one or more of the following functionalities: 1) measuring physiological data, 2) storing the measured data, 3) processing the data, 4) providing outputs (e.g., via GUIs) to a user 502 based on the processed data, and 5) communicating data with one another and/or other computing devices. Different electronic devices may perform one or more of the functionalities.

Example wearable devices 504 may include wearable computing devices, such as a ring computing device (hereinafter “ring”) configured to be worn on a user’s 502 finger, a wrist computing device (e.g., a smart watch, fitness band, or bracelet) configured to be worn on a user’s 502 wrist, and/or a head mounted computing device (e.g., glasses/goggles). Wearable devices 504 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 504 may also be attached to, or included in, articles of clothing. For example, wearable devices 504 may be included in pockets and/or pouches on clothing. As another example, wearable device 504 may be clipped and/or pinned to clothing, or may otherwise be maintained within the vicinity of the user 502. Example articles of clothing may include, but are not limited to, hats, shirts, gloves, pants, socks, outerwear (e.g., jackets), and undergarments. In some implementations, wearable devices 504 may be included with other types of devices such as training/sporting devices that are used during physical activity. For example, wearable devices 504 may be attached to, or included in, a bicycle, skis, a tennis racket, a golf club, and/or training weights.

Much of the present disclosure may be described in the context of a wearable device 504, which may include finger-worn wearable devices, wrist-worn wearable devices, and the like. Accordingly, the terms “wearable device 504,” “wearable ring device,” “ring,” and like terms, may be used interchangeably, unless noted otherwise herein. However, the use of the terms “wearable ring device” and/or “ring” are not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 506 may include handheld mobile computing devices, such as smartphones and tablet computing devices. User devices 506 may also include personal computers, such as laptop and desktop computing devices. Other example user devices 506 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 506 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 504, user devices 506) may measure physiological parameters of respective users 502, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 504), mobile device application, or a server computing device may process received physiological data that was measured by other devices.

In some implementations, a user 502 may operate, or may be associated with, multiple electronic devices, some of which may measure physiological parameters and some of which may process the measured physiological parameters. In some implementations, a user 502 may have a ring (e.g., wearable device 504) that measures physiological parameters. The user 502 may also have, or be associated with, a user device 506 (e.g., mobile device, smartphone), where the wearable device 504 and the user device 506 are communicatively coupled to one another. In some cases, the user device 506 may receive data from the wearable device 504 and perform some/all of the calculations described herein. In some implementations, the user device 506 may also measure physiological parameters described herein, such as motion/activity parameters.

For example, as illustrated in FIG. 5, a first user 502-a (User 1) may operate, or may be associated with, a wearable device 504-a (e.g., wearable ring device) and a user device 506-a that may operate as described herein. In this example, the user device 506-a associated with user 502-a may process/store physiological parameters measured by the wearable device 504-a. Comparatively, a second user 502-b (User 2) may be associated with wearable devices 504-b and 504-c (e.g., wearable ring device and a wrist-worn wearable device, such as a watch) and a user device 506-b, where the user device 506-b associated with user 502-b may process/store physiological parameters measured by the wearable devices 504-b and 504-c. Moreover, an nth user 502-n (User N) may be associated with an arrangement of electronic devices described herein (e.g., wearable device 504-n, user device 506-n). In some aspects, wearable devices 504 (e.g., wearable ring devices, wrist-worn wearable devices) and other electronic devices may be communicatively coupled to the user devices 506 of the respective users 502 via Bluetooth, Wi-Fi, and other wireless protocols. Moreover, in some cases, the wearable device 504 and the user device 506 may be included within (or make up) the same device. For example, in some cases, the wearable device 504 may be configured to execute an application associated with the wearable device 504, and may be configured to display data via a GUI.

In some implementations, the wearable devices 504 (e.g., wearable ring devices) of the system 500 may be configured to collect physiological data from the respective users 502 based on arterial blood flow within the user’s finger. In particular, a wearable ring device may utilize one or more light-emitting components, such as LEDs (e.g., red LEDs, green LEDs) that emit light on the palm-side of a user’s finger to collect physiological data based on arterial blood flow within the user’s finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 500 may be configured to collect physiological data from the respective users 502 based on blood flow diffused into a microvascular bed of skin with capillaries and arterioles. For example, the system 500 may collect PPG data based on a measured amount of blood diffused into the microvascular system of capillaries and arterioles. In some implementations, the wearable device 504 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the wearable device 504 (e.g., around an inner surface of the wearable ring device) has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a wearable ring device has been found to exhibit superior performance as compared to wearable devices worn on the wrist, as the wearable ring device may have greater access to arteries (as compared to capillaries), thereby resulting in stronger signals and more valuable physiological data.

The electronic devices of the system 500 (e.g., user devices 506, wearable devices 504) may be communicatively coupled to one or more servers 510 via wired or wireless communication protocols. For example, as shown in FIG. 5, the electronic devices (e.g., user devices 506) may be communicatively coupled to one or more servers 510 via a network 508. The network 508 may implement transfer control protocol and internet protocol (TCP/IP), such as the Internet, or may implement other network 508 protocols. Network connections between the network 508 and the respective electronic devices may facilitate transport of data via email, web, text messages, mail, or any other appropriate form of interaction within a computer network 508. For example, in some implementations, the wearable device 504-a associated with the first user 502-a may be communicatively coupled to the user device 506-a, where the user device 506-a is communicatively coupled to the servers 510 via the network 508. In additional or alternative cases, wearable devices 504 (e.g., wearable ring devices, wrist-worn wearable devices such as watches) may be directly communicatively coupled to the network 508.

The system 500 may offer an on-demand database service between the user devices 506 and the one or more servers 510. In some cases, the servers 510 may receive data from the user devices 506 via the network 508, and may store and analyze the data. Similarly, the servers 510 may provide data to the user devices 506 via the network 508. In some cases, the servers 510 may be located at one or more data centers. The servers 510 may be used for data storage, management, and processing. In some implementations, the servers 510 may provide a web-based interface to the user device 506 via web browsers.

In some aspects, the system 500 may detect periods of time that a user 502 is asleep, and classify periods of time that the user 502 is asleep into one or more sleep stages (e.g., sleep stage classification). For example, as shown in FIG. 5, User 502-a may be associated with a wearable device 504-a (e.g., wearable ring device) and a user device 506-a. In this example, the wearable device 504-a may collect physiological data associated with the user 502-a, including temperature, heart rate, HRV, respiratory rate, and the like. In some aspects, data collected by the wearable device 504-a may be input to a machine learning classifier, where the machine learning classifier is configured to determine periods of time that the user 502-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 502-a via a GUI of the user device 506-a. Sleep stage classification may be used to provide feedback to a user 502-a regarding the user’s sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 500 may utilize circadian rhythm-derived features to further improve physiological data collection, data processing procedures, and other techniques described herein. The term circadian rhythm may refer to a natural, internal process that regulates an individual’s sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 502-a via the wearable device 504-a. In this example, the circadian rhythm adjustment model may be configured to “weight,” or adjust, physiological data collected throughout a user’s natural, approximately 24-hour circadian rhythm. In some implementations, the system may initially start with a “baseline” circadian rhythm adjustment model, and may modify the baseline model using physiological data collected from each user 502 to generate tailored, individualized circadian rhythm adjustment models that are specific to each respective user 502.

In some aspects, the system 500 may utilize other biological rhythms to further improve physiological data collection, analysis, and processing by phase of these other rhythms. For example, if a weekly rhythm is detected within an individual’s baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 500 may support dome-shaped protrusions for wearable devices 104 that are formed from a glass material. For example, the glass may be formed into a dome shape and sealed to a housing of the wearable device to form a relatively more robust hollow dome-shaped protrusion that may result in relatively higher quality of measurements (e.g., as compared to some other optically transparent materials, such as epoxy). In some examples, the dome-shaped protrusions may be formed from a sheet of glass that is curved into a molded shape or from an unmolded glass preform that is pressed into the molded shape. In some examples, the glass may be bonded with the housing of the wearable device during the molding process (e.g., with the housing forming a portion of the mold for the dome-shaped protrusion) or following the molding process.

It should be appreciated by a person skilled in the art that one or more aspects of the disclosure may be implemented in a system 500 to additionally, or alternatively, solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

FIG. 6 illustrates an example of a system 600 that supports techniques for forming glass domes for wearable devices in accordance with aspects of the present disclosure. The system 600 may implement, or be implemented by, system 500. In particular, system 600 illustrates a wearable device 604 (e.g., wearable ring device), a user device 606, and a server 610, as described with reference to FIG. 5.

In some aspects, the wearable device 604 (e.g., wearable ring device) may be configured to be worn around a user’s finger, and may determine one or more user physiological parameters when worn around the user’s finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 600 further includes a user device 606 (e.g., a smartphone) in communication with the wearable device 604. For example, the wearable device 604 may be in wireless and/or wired communication with the user device 606. In some implementations, the wearable device 604 may send measured and processed data (e.g., temperature data, photoplethysmogram (PPG) data, motion/accelerometer data, ring input data, and the like) to the user device 606. The user device 606 may also send data to the wearable device 604, such as firmware/configuration updates. The user device 606 may process data. In some implementations, the user device 606 may transmit data to the server 610 for processing and/or storage.

The wearable device 604 may include a housing 605 that may include an inner housing 605-a and an outer housing 605-b. In some aspects, the inner housing 605-a, the outer housing 605-b, or both, may include a curved profile/surface. In particular, the housing 605 may exhibit any curved or “circumferential” profile, including a circular profile, an elliptical profile, and the like. Moreover, in some cases, the inner housing 605-a, the outer housing 605-b, or both, may include both curved (e.g., “circumferential”) and flat/planar portions. For the purposes of the present disclosure, the term “circumferential” may be used interchangeably with the term “curved” to refer to circular-shaped, elliptical-shaped, or other curved-shaped profile.

In some aspects, the housing 605 of the wearable device 604 may store or otherwise include various components of the ring including, but not limited to, device electronics, a power source (e.g., battery 611, and/or capacitor), one or more substrates (e.g., printable circuit boards) that interconnect the device electronics and/or power source, and the like. The device electronics may include device modules (e.g., hardware/software), such as: a processing module 630-a, a memory 615, a communication module 620-a, a power module 625, and the like. The device electronics may also include one or more sensors. Example sensors may include one or more temperature sensors 640, a PPG sensor assembly (e.g., PPG system 635), and one or more motion sensors 645.

The sensors may include associated modules (not illustrated) configured to communicate with the respective components/modules of the wearable device 604, and generate signals associated with the respective sensors. In some aspects, each of the components/modules of the wearable device 604 may be communicatively coupled to one another via wired or wireless connections. Moreover, the wearable device 604 may include additional and/or alternative sensors or other components that are configured to collect physiological data from the user, including light sensors (e.g., LEDs), oximeters, and the like.

The wearable device 604 shown and described with reference to FIG. 6 is provided solely for illustrative purposes. As such, the wearable device 604 may include additional or alternative components as those illustrated in FIG. 6. Additional or alternative wearable devices 604 that provide functionality described herein may be fabricated. For example, wearable devices 604 with fewer components (e.g., sensors) may be fabricated. In a specific example, a wearable device 604 with a single temperature sensor 640 (or other sensor), a power source, and device electronics configured to read the single temperature sensor 640 (or other sensor) may be fabricated. In another specific example, a temperature sensor 640 (or other sensor) may be attached to a user’s finger (e.g., using adhesives, wraps, clamps, spring loaded clamps, etc.). In this case, the sensor may be wired to another computing device, such as a wrist worn computing device that reads the temperature sensor 640 (or other sensor). In other examples, a wearable device 604 that includes additional sensors and processing functionality may be fabricated.

The housing 605 may include one or more housing components. The housing 605 may include an outer housing 605-b component (e.g., a shell) and an inner housing 605-a component (e.g., a molding). The housing 605 may include additional components (e.g., additional layers) not explicitly illustrated in FIG. 6. For example, in some implementations, the wearable device 604 may include one or more insulating layers that electrically insulate the device electronics and other conductive materials (e.g., electrical traces) from the outer housing 605-b. The housing 605 may provide structural support for the device electronics, battery 611, substrate(s), and other components. For example, the housing 605 may protect the device electronics, battery 611, and substrate(s) from mechanical forces, such as pressure and impacts. The housing 605 may also protect the device electronics, battery 611, and substrate(s) from water and/or other chemicals.

The inner housing 605-a may be configured to interface with the user’s finger. The inner housing 605-a may be formed from a polymer (e.g., a medical grade polymer) or other material. In some implementations, the inner housing 605-a may be transparent. For example, the inner housing 605-a may be transparent to light emitted by the PPG LEDs. In some implementations, the inner housing 605-a component may be molded onto the outer housing 605-b. For example, the inner housing 605-a may include a polymer that is molded (e.g., injection molded) to fit into an outer housing 605-b metallic shell.

The inner housing 605-a and the outer housing 605-b may be fabricated from one or more materials. In some implementations, the inner housing 605-a, the outer housing 605-b, or both, may include a metal, such as titanium, that may provide strength and abrasion resistance at a relatively light weight. Additionally, or alternatively, the inner housing 605-a, and/or the outer housing 605-b may also be fabricated from other materials, such polymers, plastic materials, epoxy materials, ceramic materials, and the like. In some implementations, the outer housing 605-b may be protective as well as decorative.

The wearable device 604 may include one or more substrates (not illustrated). The device electronics and battery 611 may be included on the one or more substrates. For example, the device electronics and battery 611 may be mounted on one or more substrates. Example substrates may include one or more printed circuit boards (PCBs), such as flexible PCB (e.g., polyimide). In some implementations, the electronics/battery 611 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 611 to the device electronics.

The device electronics, battery 611, and substrates may be arranged in the wearable device 604 in a variety of ways. In some implementations, one substrate that includes device electronics may be mounted along the bottom of the wearable device 604 (e.g., the bottom half), such that the sensors (e.g., PPG system 635, temperature sensors 640, motion sensors 645, and other sensors) interface with the underside of the user’s finger. In these implementations, the battery 611 may be included along the top portion of the wearable device 604 (e.g., on another substrate).

The various components/modules of the wearable device 604 represent functionality (e.g., circuits and other components) that may be included in the wearable device 604. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 615 (memory module) of the wearable device 604 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 615 may store any of the data described herein. For example, the memory 615 may be configured to store data (e.g., motion data, temperature data, PPG data) collected by the respective sensors and PPG system 635. Furthermore, memory 615 may include instructions that, when executed by one or more processing circuits, cause the modules to perform various functions attributed to the modules herein. The device electronics of the wearable device 604 described herein are only example device electronics. As such, the types of electronic components used to implement the device electronics may vary based on design considerations.

The functions attributed to the modules of the wearable device 604 (e.g., wearable ring device) described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 630-a of the wearable device 604 may include one or more processors (e.g., processing units), microcontrollers, digital signal processors, systems on a chip (SOCs), and/or other processing devices. The processing module 630-a communicates with the modules included in the wearable device 604. For example, the processing module 630-a may transmit/receive data to/from the modules and other components of the wearable device 604, such as the sensors. As described herein, the modules may be implemented by various circuit components. Accordingly, the modules may also be referred to as circuits (e.g., a communication circuit and power circuit).

The processing module 630-a may communicate with the memory 615. The memory 615 may include computer-readable instructions that, when executed by the processing module 630-a, cause the processing module 630-a to perform the various functions attributed to the processing module 630-a herein. In some implementations, the processing module 630-a (e.g., a microcontroller) may include additional features associated with other modules, such as communication functionality provided by the communication module 620-a (e.g., an integrated Bluetooth Low Energy transceiver) and/or additional onboard memory 615.

The communication module 620-a may include circuits that provide wireless and/or wired communication with the user device 606 (e.g., communication module 620-b of the user device 606). In some implementations, the communication modules 620-a, 620-b may include wireless communication circuits, such as Bluetooth circuits and/or Wi-Fi circuits. In some implementations, the communication modules 620-a, 620-b can include wired communication circuits, such as Universal Serial Bus (USB) communication circuits. Using the communication module 620-a, the wearable device 604 and the user device 606 may be configured to communicate with each other. The processing module 630-a of the ring may be configured to transmit/receive data to/from the user device 606 via the communication module 620-a. Example data may include, but is not limited to, motion data, temperature data, pulse waveforms, heart rate data, HRV data, PPG data, and status updates (e.g., charging status, battery charge level, and/or wearable device 604 configuration settings). The processing module 630-a of the ring may also be configured to receive updates (e.g., software/firmware updates) and data from the user device 606.

The wearable device 604 may include a battery 611 (e.g., a rechargeable battery 611). An example battery 611 may include a Lithium-Ion or Lithium-Polymer type battery 611, although a variety of battery 611 options are possible. The battery 611 may be wirelessly charged. In some implementations, the wearable device 604 may include a power source other than the battery 611, such as a capacitor. The power source (e.g., battery 611 or capacitor) may have a curved geometry that matches the curve of the wearable device 604. In some aspects, a charger or other power source may include additional sensors that may be used to collect data in addition to, or that supplements, data collected by the wearable device 604 itself. Moreover, a charger or other power source for the wearable device 604 may function as a user device 606, in which case the charger or other power source for the wearable device 604 may be configured to receive data from the wearable device 604, store and/or process data received from the wearable device 604, and communicate data between the wearable device 604 and the servers 610.

In some aspects, the wearable device 604 includes a power module 625 that may control charging of the battery 611. For example, the power module 625 may interface with an external wireless charger that charges the battery 611 when interfaced with the wearable device 604. The charger may include a datum structure that mates with a wearable device 604 datum structure to create a specified orientation with the wearable device 604 during charging. The power module 625 may also regulate voltage(s) of the device electronics, regulate power output to the device electronics, and monitor the state of charge of the battery 611. In some implementations, the battery 611 may include a protection circuit module (PCM) that protects the battery 611 from high current discharge, over voltage during charging, and under voltage during discharge. The power module 625 may also include electro-static discharge (ESD) protection.

The one or more temperature sensors 640 may be electrically coupled to the processing module 630-a. The temperature sensor 640 may be configured to generate a temperature signal (e.g., temperature data) that indicates a temperature read or sensed by the temperature sensor 640. The processing module 630-a may determine a temperature of the user in the location of the temperature sensor 640. For example, in the wearable device 604, temperature data generated by the temperature sensor 640 may indicate a temperature of a user at the user’s finger (e.g., skin temperature). In some implementations, the temperature sensor 640 may contact the user’s skin. In other implementations, a portion of the housing 605 (e.g., the inner housing 605-a) may form a barrier (e.g., a thin, thermally conductive barrier) between the temperature sensor 640 and the user’s skin. In some implementations, portions of the wearable device 604 configured to contact the user’s finger may have thermally conductive portions and thermally insulative portions. The thermally conductive portions may conduct heat from the user’s finger to the temperature sensors 640. The thermally insulative portions may insulate portions of the wearable device 604 (e.g., the temperature sensor 640) from ambient temperature.

In some implementations, the temperature sensor 640 may generate a digital signal (e.g., temperature data) that the processing module 630-a may use to determine the temperature. As another example, in cases where the temperature sensor 640 includes a passive sensor, the processing module 630-a (or a temperature sensor 640 module) may measure a current/voltage generated by the temperature sensor 640 and determine the temperature based on the measured current/voltage. Example temperature sensors 640 may include a thermistor, such as a negative temperature coefficient (NTC) thermistor, or other types of sensors including resistors, transistors, diodes, and/or other electrical/electronic components.

The processing module 630-a may sample the user’s temperature over time. For example, the processing module 630-a may sample the user’s temperature according to a sampling rate. An example sampling rate may include one sample per second, although the processing module 630-a may be configured to sample the temperature signal at other sampling rates that are higher or lower than one sample per second. In some implementations, the processing module 630-a may sample the user’s temperature continuously throughout the day and night. Sampling at a sufficient rate (e.g., one sample per second) throughout the day may provide sufficient temperature data for analysis described herein.

The processing module 630-a may store the sampled temperature data in memory 615. In some implementations, the processing module 630-a may process the sampled temperature data. For example, the processing module 630-a may determine average temperature values over a period of time. In one example, the processing module 630-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 615 may store the average temperature values over time. In some implementations, the memory 615 may store average temperatures (e.g., one per minute) instead of sampled temperatures in order to conserve memory 615.

The sampling rate, which may be stored in memory 615, may be configurable. In some implementations, the sampling rate may be the same throughout the day and night. In other implementations, the sampling rate may be changed throughout the day/night. In some implementations, the wearable device 604 may filter/reject temperature readings, such as large spikes in temperature that are not indicative of physiological changes (e.g., a temperature spike from a hot shower). In some implementations, the wearable device 604 may filter/reject temperature readings that may not be reliable due to other factors, such as excessive motion during exercise (e.g., as indicated by a motion sensor 645).

The wearable device 604 (e.g., communication module) may transmit the sampled and/or average temperature data to the user device 606 for storage and/or further processing. The user device 606 may transfer the sampled and/or average temperature data to the server 610 for storage and/or further processing.

Although the wearable device 604 is illustrated as including a single temperature sensor 640, the wearable device 604 may include multiple temperature sensors 640 in one or more locations, such as arranged along the inner housing 605-a near the user’s finger. In some implementations, the temperature sensors 640 may be stand-alone temperature sensors 640. Additionally, or alternatively, one or more temperature sensors 640 may be included with other components (e.g., packaged with other components), such as with the accelerometer and/or processor.

The processing module 630-a may acquire and process data from multiple temperature sensors 640 in a similar manner described with respect to a single temperature sensor 640. For example, the processing module 630 may individually sample, average, and store temperature data from each of the multiple temperature sensors 640. In other examples, the processing module 630-a may sample the sensors at different rates and average/store different values for the different sensors. In some implementations, the processing module 630-a may be configured to determine a single temperature based on the average of two or more temperatures determined by two or more temperature sensors 640 in different locations on the finger.

The temperature sensors 640 on the wearable device 604 (e.g., wearable ring device) may acquire distal temperatures at the user’s finger (e.g., any finger). For example, one or more temperature sensors 640 on the wearable device 604 may acquire a user’s temperature from the underside of a finger or at a different location on the finger. In some implementations, the wearable device 604 may continuously acquire distal temperature (e.g., at a sampling rate). Although distal temperature measured by a wearable device 604 at the finger is described herein, other devices may measure temperature at the same/different locations. In some cases, the distal temperature measured at a user’s finger may differ from the temperature measured at a user’s wrist or other external body location. Additionally, the distal temperature measured at a user’s finger (e.g., a “shell” temperature) may differ from the user’s core temperature. As such, the wearable device 604 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The wearable device 604 may include a PPG system 635. The PPG system 635 may include one or more optical transmitters that transmit light. The PPG system 635 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user’s finger. The PPG signal generated by the PPG system 635 may indicate the perfusion of blood in the illuminated region. For example, the PPG signal may indicate blood volume changes in the illuminated region caused by a user’s pulse pressure. The processing module 630-a may sample the PPG signal and determine a user’s pulse waveform based on the PPG signal. The processing module 630-a may determine a variety of physiological parameters based on the user’s pulse waveform, such as a user’s respiratory rate, heart rate, HRV, oxygen saturation, and other circulatory parameters.

In some implementations, the PPG system 635 may be configured as a reflective PPG system 635 where the optical receiver(s) receive transmitted light that is reflected through the region of the user’s finger. In some implementations, the PPG system 635 may be configured as a transmissive PPG system 635 where the optical transmitter(s) and optical receiver(s) are arranged opposite to one another, such that light is transmitted directly through a portion of the user’s finger to the optical receiver(s).

The number and ratio of transmitters and receivers included in the PPG system 635 may vary. Example optical transmitters may include LEDs. The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 635.

The PPG system 635 illustrated in FIG. 6 may include a reflective PPG system 635 in some implementations. In these implementations, the PPG system 635 may include a centrally located optical receiver (e.g., at the bottom of the wearable device 604) and two optical transmitters located on each side of the optical receiver. In this implementation, the PPG system 635 (e.g., optical receiver) may generate the PPG signal based on light received from one or both of the optical transmitters. In other implementations, other placements, combinations, and/or configurations of one or more optical transmitters and/or optical receivers are contemplated.

The processing module 630-a may control one or both of the optical transmitters to transmit light while sampling the PPG signal generated by the optical receiver. In some implementations, the processing module 630-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 635 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 630-a may store the pulse waveform in memory 615 in some implementations. The processing module 630-a may process the pulse waveform as it is generated and/or from memory 615 to determine user physiological parameters described herein.

The processing module 630-a may determine the user’s heart rate based on the pulse waveform. For example, the processing module 630-a may determine heart rate (e.g., in beats per minute) based on the time between peaks in the pulse waveform. The time between peaks may be referred to as an interbeat interval (IBI). The processing module 630-a may store the determined heart rate values and IBI values in memory 615.

The processing module 630-a may determine HRV over time. For example, the processing module 630-a may determine HRV based on the variation in the IBIs. The processing module 630-a may store the HRV values over time in the memory 615. Moreover, the processing module 630-a may determine the user’s respiratory rate over time. For example, the processing module 630-a may determine respiratory rate based on frequency modulation, amplitude modulation, or baseline modulation of the user’s IBI values over a period of time. Respiratory rate may be calculated in breaths per minute or as another breathing rate (e.g., breaths per 30 seconds). The processing module 630-a may store user respiratory rate values over time in the memory 615.

The wearable device 604 may include one or more motion sensors 645, such as one or more accelerometers (e.g., 6-D accelerometers) and/or one or more gyroscopes (gyros). The motion sensors 645 may generate motion signals that indicate motion of the sensors. For example, the wearable device 604 may include one or more accelerometers that generate acceleration signals that indicate acceleration of the accelerometers. As another example, the wearable device 604 may include one or more gyro sensors that generate gyro signals that indicate angular motion (e.g., angular velocity) and/or changes in orientation. The motion sensors 645 may be included in one or more sensor packages. An example accelerometer/gyro sensor is a Bosch BMI160 inertial micro electro-mechanical system (MEMS) sensor that may measure angular rates and accelerations in three perpendicular axes.

The processing module 630-a may sample the motion signals at a sampling rate (e.g., 50Hz) and determine the motion of the wearable device 604 based on the sampled motion signals. For example, the processing module 630-a may sample acceleration signals to determine acceleration of the wearable device 604. As another example, the processing module 630-a may sample a gyro signal to determine angular motion. In some implementations, the processing module 630-a may store motion data in memory 615. Motion data may include sampled motion data as well as motion data that is calculated based on the sampled motion signals (e.g., acceleration and angular values).

The wearable device 604 may store a variety of data described herein. For example, the wearable device 604 may store temperature data, such as raw sampled temperature data and calculated temperature data (e.g., average temperatures). As another example, wearable device 604 may store PPG signal data, such as pulse waveforms and data calculated based on the pulse waveforms (e.g., heart rate values, IBI values, HRV values, and respiratory rate values). The wearable device 604 may also store motion data, such as sampled motion data that indicates linear and angular motion.

The wearable device 604, or other computing device, may calculate and store additional values based on the sampled/calculated physiological data. For example, the processing module 630 may calculate and store various metrics, such as sleep metrics (e.g., a Sleep Score), activity metrics, and readiness metrics. In some implementations, additional values/metrics may be referred to as “derived values.” The wearable device 604, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the wearable device 604 is oriented on the user’s finger and if the wearable device 604 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 630-a may compress the data stored in memory 615. For example, the processing module 630-a may delete sampled data after making calculations based on the sampled data. As another example, the processing module 630-a may average data over longer periods of time in order to reduce the number of stored values. In a specific example, if average temperatures for a user over one minute are stored in memory 615, the processing module 630-a may calculate average temperatures over a five minute time period for storage, and then subsequently erase the one minute average temperature data. The processing module 630-a may compress data based on a variety of factors, such as the total amount of used/available memory 615 and/or an elapsed time since the wearable device 604 last transmitted the data to the user device 606.

Although a user’s physiological parameters may be measured by sensors included on a wearable device 604, other devices may measure a user’s physiological parameters. For example, although a user’s temperature may be measured by a temperature sensor 640 included in a wearable device 604, other devices may measure a user’s temperature. In some examples, other wearable devices (e.g., wrist devices) may include sensors that measure user physiological parameters. Additionally, medical devices, such as external medical devices (e.g., wearable medical devices) and/or implantable medical devices, may measure a user’s physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the wearable device 604 can make physiological measurements in a resting/sleep state in order to acquire cleaner physiological signals. In one example, the wearable device 604 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the wearable device 604 may be configured to collect, store, and/or process data, and may transfer any of the data described herein to the user device 606 for storage and/or processing. In some aspects, the user device 606 includes a wearable application 650, an operating system 685 (OS), a web browser application (e.g., web browser 680), one or more additional applications, and a GUI 675. The user device 606 may further include other modules and components, including sensors, audio devices, haptic feedback devices, and the like. The wearable application 650 may include an example of an application (e.g., “app”) that may be installed on the user device 606. The wearable application 650 may be configured to acquire data from the wearable device 604, store the acquired data, and process the acquired data as described herein. For example, the wearable application 650 may include a user interface (UI) module 655, an acquisition module 660, a processing module 630-b, a communication module 620-b, and a storage module (e.g., database 665) configured to store application data.

In some cases, the wearable device 604 and the user device 606 may be included within (or make up) the same device. For example, in some cases, the wearable device 604 may be configured to execute the wearable application 650, and may be configured to display data via the GUI 675.

The various data processing operations described herein may be performed by the wearable device 604, the user device 606, the servers 610, or any combination thereof. For example, in some cases, data collected by the wearable device 604 may be pre-processed and transmitted to the user device 606. In this example, the user device 606 may perform some data processing operations on the received data, may transmit the data to the servers 610 for data processing, or both. For instance, in some cases, the user device 606 may perform processing operations that require relatively low processing power and/or operations that require a relatively low latency, whereas the user device 606 may transmit the data to the servers 610 for processing operations that require relatively high processing power and/or operations that may allow relatively higher latency.

In some aspects, the wearable device 604 (e.g., wearable ring device), user device 606, and server 610 of the system 600 may be configured to evaluate sleep patterns for a user. In particular, the respective components of the system 600 may be used to collect data from a user via the wearable device 604, and generate one or more scores (e.g., Sleep Score, Readiness Score) for the user based on the collected data. For example, as noted previously herein, the wearable device 604 of the system 600 may be worn by a user to collect data from the user, including temperature, heart rate, HRV, and the like. Data collected by the wearable device 604 may be used to determine when the user is asleep in order to evaluate the user’s sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the wearable device 604 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 600 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user’s overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user’s sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user’s overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user’s needs. Typically, adults need 7–9 hours of sleep a night to stay healthy, alert, and to perform at their best both mentally and physically. However, it is normal to have an occasional night of bad sleep, so the sleep balance contributor takes into account long-term sleep patterns to determine whether each user’s sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user’s resting heart rate to stabilize during the night. A sign of a very good recovery is that the user’s resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user’s highest temperature during the nap is at least 0.5°C higher than the highest temperature during the longest period. In some aspects, the ring may measure a user’s body temperature while the user is asleep, and the system 600 may display the user’s average temperature relative to the user’s baseline temperature. If a user’s body temperature is outside of their normal range (e.g., clearly above or below 0.0), the body temperature contributor may be highlighted (e.g., go to a “Pay attention” state) or otherwise generate an alert for the user.

In some aspects, the system 600 may support dome-shaped protrusions for wearable devices 104 that are formed from a glass material. For example, the glass may be formed into a dome shape and sealed to a housing of the wearable device to form a relatively more robust hollow dome-shaped protrusion that may result in relatively higher quality of measurements (e.g., as compared to some other optically transparent materials, such as epoxy). In some examples, the dome-shaped protrusions may be formed from a sheet of glass that is curved into a molded shape or from an unmolded glass preform that is pressed into the molded shape. In some examples, the glass may be bonded with the housing of the wearable device during the molding process (e.g., with the housing forming a portion of the mold for the dome-shaped protrusion) or following the molding process.

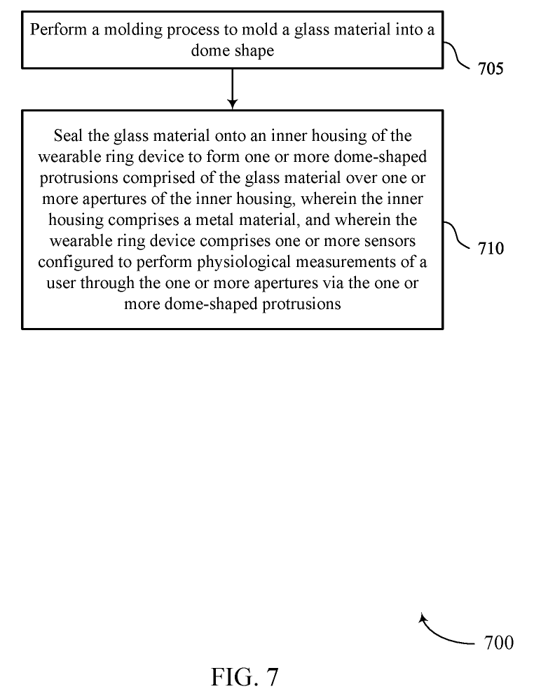

FIG. 7 shows a flowchart illustrating a method 700 that supports techniques for forming glass domes for wearable devices in accordance with aspects of the present disclosure. The operations of the method 700 may be implemented by a wearable device or its components as described herein. For example, the operations of the method 700 may be performed by a wearable device as described with reference to FIGs. 1 through 6. In some examples, a wearable device may execute a set of instructions to control the functional elements of the wearable device to perform the described functions. Additionally, or alternatively, the wearable device may perform aspects of the described functions using special-purpose hardware.

At 705, the method may include performing a molding process to mold a glass material into a dome shape. The operations of 705 may be performed in accordance with examples as disclosed herein.

At 710, the method may include sealing the glass material onto an inner housing of the wearable ring device to form one or more dome-shaped protrusions comprised of the glass material over one or more apertures of the inner housing, wherein the inner housing comprises a metal material, and wherein the wearable ring device comprises one or more sensors configured to perform physiological measurements of a user through the one or more apertures via the one or more dome-shaped protrusions. The operations of 710 may be performed in accordance with examples as disclosed herein.

It should be noted that the methods described above describe possible implementations, and that the operations and the steps may be rearranged or otherwise modified and that other implementations are possible. Furthermore, aspects from two or more of the methods may be combined.

The following provides an overview of aspects of the present disclosure:

Aspect 1: A wearable ring device, comprising: an inner housing comprising one or more apertures, wherein the inner housing comprises a metal material; one or more dome-shaped protrusions positioned over the one or more apertures and sealed to the metal material of the inner housing, wherein the one or more dome-shaped protrusions comprise a glass material and form one or more air-filled cavities corresponding to the one or more apertures; and one or more sensors positioned within the wearable ring device to transmit or receive light through the one or more apertures and the one or more dome-shaped protrusions.

Aspect 2: The wearable ring device of aspect 1, wherein the one or more dome-shaped protrusions are sealed to the metal material of the inner housing via an adhesive material.

Aspect 3: The wearable ring device of any of aspects 1 through 2, wherein the one or more dome-shaped protrusions are hermetically sealed to the metal material of the inner housing.

Aspect 4: The wearable ring device of any of aspects 1 through 3, wherein the glass material is formed from a sheet of glass.

Aspect 5: The wearable ring device of aspect 4, wherein the one or more dome-shaped protrusions comprise a plurality of dome-shaped protrusions formed from a continuous sheet of glass.

Aspect 6: The wearable ring device of any of aspects 1 through 5, wherein the glass material is formed from a glass preform.

Aspect 7: The wearable ring device of aspect 6, wherein the one or more dome-shaped protrusions comprise a plurality of dome-shaped protrusions formed from a single glass preform.

Aspect 8: The wearable ring device of any of aspects 1 through 7, wherein the metal material is titanium, stainless steel, or any combination thereof.

Aspect 9: The wearable ring device of any of aspects 1 through 8, wherein the wearable ring device is configured to perform physiological measurements of a user via the one or more sensors.

Aspect 10: The wearable ring device of any of aspects 1 through 9, wherein the one or more sensors comprise a first light-emitting component housed within a first dome-shaped protrusion of the one or more dome-shaped protrusions and a second light-emitting component housed within a second dome-shaped protrusion of the one or more dome-shaped protrusions.

Aspect 11: A method for manufacturing a wearable ring device, comprising: performing a molding process to mold a glass material into a dome shape; and sealing the glass material onto an inner housing of the wearable ring device to form one or more dome-shaped protrusions comprised of the glass material over one or more apertures of the inner housing, wherein the inner housing comprises a metal material, and wherein the wearable ring device comprises one or more sensors configured to perform physiological measurements of a user through the one or more apertures via the one or more dome-shaped protrusions.

Aspect 12: The method of aspect 11, wherein the molding process comprises: placing a sheet of the glass material into a mold; heating the sheet of the glass material; and pressing the sheet of glass into a mold to form the one or more dome-shaped protrusions.

Aspect 13: The method of aspect 12, wherein sealing the glass material onto the inner housing of the wearable ring device comprises: applying an adhesive material to the dome shape formed from the sheet of the glass material; and adhering the glass material onto the inner housing of the wearable ring device via the adhesive material.

Aspect 14: The method of any of aspects 11 through 13, wherein the molding process comprises: placing a preform of the glass material into a mold; heating the preform of the glass material; and pressing the preform of glass into a mold to form the one or more dome-shaped protrusions, wherein a portion of the mold is formed from the inner housing, and wherein sealing the glass material onto the inner housing comprises hermetically sealing the glass material onto the inner housing.

Aspect 15: The method of any of aspects 11 through 14, wherein performing the molding process comprises: performing the molding process to mold the glass material into a plurality of dome shapes.

Aspect 16: The method of any of aspects 11 through 15, wherein the metal material of the inner housing is titanium, stainless steel, or any combination thereof.

Aspect 17: The method of any of aspects 11 through 16, wherein the one or more sensors comprise a first light-emitting component housed within a first dome-shaped protrusion of the one or more dome-shaped protrusions and a second light-emitting component housed within a second dome-shaped protrusion of the one or more dome-shaped protrusions.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A wearable ring device, comprising:
an inner housing comprising one or more apertures, wherein the inner housing comprises a metal material; one or more dome-shaped protrusions positioned over the one or more apertures and sealed to the metal material of the inner housing, wherein the one or more dome-shaped protrusions comprise a glass material and form one or more air-filled cavities corresponding to the one or more apertures; and one or more sensors positioned within the wearable ring device to transmit or receive light through the one or more apertures and the one or more dome-shaped protrusions.

2. The wearable ring device of claim 1, wherein the one or more dome-shaped protrusions are sealed to the metal material of the inner housing via an adhesive material.

3. The wearable ring device of claim 1, wherein the one or more dome-shaped protrusions are hermetically sealed to the metal material of the inner housing.

4. The wearable ring device of claim 1, wherein the glass material is formed from a sheet of glass.

5. The wearable ring device of claim 4, wherein the one or more dome-shaped protrusions comprise a plurality of dome-shaped protrusions formed from a continuous sheet of glass.

6. The wearable ring device of claim 1, wherein the glass material is formed from a glass preform.

7. The wearable ring device of claim 6, wherein the one or more dome-shaped protrusions comprise a plurality of dome-shaped protrusions formed from a single glass preform.

8. The wearable ring device of claim 1, wherein the metal material comprises titanium, stainless steel, or any combination thereof.

9. The wearable ring device of claim 1, wherein the wearable ring device is configured to perform physiological measurements of a user via the one or more sensors.

10. The wearable ring device of claim 1, wherein the one or more sensors comprise a first light-emitting component positioned at least partially within a first dome-shaped protrusion of the one or more dome-shaped protrusions, and a second light-emitting component positioned at least partially within a second dome-shaped protrusion of the one or more dome-shaped protrusions.

11. A method for manufacturing a wearable ring device, comprising:
performing a molding process to mold a glass material into a dome shape; and sealing the glass material onto an inner housing of the wearable ring device to form one or more dome-shaped protrusions comprised of the glass material over one or more apertures of the inner housing, wherein the inner housing comprises a metal material, and wherein the wearable ring device comprises one or more sensors configured to perform physiological measurements of a user through the one or more apertures via the one or more dome-shaped protrusions.

12. The method of claim 11, wherein the molding process comprises:
placing a sheet of the glass material into a mold; heating the sheet of the glass material; and pressing the sheet of glass into a mold to form the one or more dome-shaped protrusions.

13. The method of claim 12, wherein sealing the glass material onto the inner housing of the wearable ring device comprises:
applying an adhesive material to the dome shape formed from the sheet of the glass material; and adhering the glass material onto the inner housing of the wearable ring device via the adhesive material.

14. The method of claim 11, wherein the molding process comprises:
placing a preform of the glass material into a mold; heating the preform of the glass material; and pressing the preform of glass into a mold to form the one or more dome-shaped protrusions, wherein a portion of the mold is formed from the inner housing, and wherein sealing the glass material onto the inner housing comprises hermetically sealing the glass material onto the inner housing.

15. The method of claim 11, wherein performing the molding process comprises:
performing the molding process to mold the glass material into a plurality of dome shapes.

16. The method of claim 11, wherein the metal material of the inner housing comprises titanium, stainless steel, or any combination thereof.

17. The method of claim 11, wherein the one or more sensors comprise a first light-emitting component positioned at least partially within a first dome-shaped protrusion of the one or more dome-shaped protrusions, and a second light-emitting component positioned at least partially within a second dome-shaped protrusion of the one or more dome-shaped protrusions.
